(19) United States
(12) Patent Application Publication
(10) Pub. No.: US 2003/0191418 A1
(43) Pub. Date: Oct. 9, 2003

(54) Breast and bottle feeding infant head support

(76) Inventor: Elizabeth M. Pillari, Tinton Falls, NJ (US)

Correspondence Address: Charles I. Brodsky, 2 Bucks Lane, Marlboro, NJ, 07746, US

(21) Appl. No.: 10/115,068

(22) Filed: Apr. 4, 2002

(51) Int. Cl. A61F 13/06; A61F 13/00; A61F 5/00

(52) U.S. Cl. 602/3; 602/63

(57) ABSTRACT

A breast and bottle feeding infant head support including a slip-on sleeve open at opposite ends and inclusive of inner and outer linings forming first and second opposing face surfaces of predetermined length and width, and a cushion insert between the inner and outer linings beneath at least one of the opposing face surfaces wherein the sleeve, when worn on the arm, is of a length to extend past the crook of a wearer's elbow.

### STATEMENT REGARDING FEDERALLY SPONSORED RESEARCH OR DEVELOPMENT

Research and development of this invention and Application have not been federally sponsored, and no rights are given under any Federal program.

### CROSS-REFERENCE TO RELATED APPLICATIONS

NONE

### REFERENCE TO A MICROFICHE APPENDIX

NOT APPLICABLE

### BACKGROUND OF THE INVENTION

1. Field of the Invention

This invention relates to the breast and bottle feeding of infants, in general, and to a support to make breast and bottle feeding comfortable for both a mother and her baby, in particular.

2. Description of the Related Art

As is well known and understood, in the breast and/or bottle feeding of infants, the baby's head is typically supported against the mother's inside forearm. Experience has shown that with the forearm being firmer than the baby's head, these breast and bottle feedings over time tend to somewhat flatten the head of the infant. Such head, besides being so malleable, also tends to absorb the perspiration built up on the forearm during hot humid weather, especially when the mother wears a short sleeve blouse. These deficiencies and detriments are addressed by the present invention.

### SUMMARY OF THE INVENTION

As will become clear from the following description, the present invention comprises a breast and bottle feeding infant head support utilizing a slip-on sleeve open at the opposite ends thereof, and inclusive of inner and outer linings forming first and second opposing face surfaces of predetermined length and width. A cushion insert is added according to the invention, between the inner and outer linings beneath one of the opposing face surfaces. When the sleeve is worn on the arm, and selected of a length to extend past the crook of a wearer's elbow, the cushion support gives to the sleeve a padded feeling providing a comfort both for the mother and for her baby. In a preferred embodiment to be described, the slip-on sleeve is of a durable fabric-material, softly absorbent, and washable—as of a cotton fabric, for example. At the same time, the cushion insert is preferably resilient, as a foam, for instance. Selected of a width substantially equal to the predetermined widths of the first and second opposing face surfaces, the cushion insert of the invention could similarly be selected of a length substantially equal to the predetermined lengths of the first and second opposing surfaces, or of a length somewhat less. In such latter instance, the slip-on sleeve may extend from past the crook of the elbow towards the palm of the hand, where the fingers could encircle it to hold it in place, without grasping onto the cushion insert.

In a further embodiment of the invention, a second insert may be added, between the inner and outer linings beneath the other of the two opposing face surfaces, so that the slip-on sleeve could be alternated in wearing with first one, then the other, surface supporting the infant's head during alternative feedings. There, too, the second cushion insert could be of comparable lengths and widths to that of the face surfaces, or of differing length surface if so desired.

As will be appreciated, a cradle essentially is established for supporting the infant's head, in a manner to absorb perspiration, with a soft absorbent fabric, and in a manner to make the breast and bottle feeding a comfort not only for the infant; but for the mother, as well.

### BRIEF DESCRIPTION OF THE DRAWINGS

These and other features of the present invention will be more clearly understood from a consideration of the following description, taken in connection with the accompanying drawings, in which:

FIG. 1 is a perspective view of a breast and bottle feeding infant head support embodying the invention; and

FIGS. 2 and 3 are partial perspective views helpful in an understanding of preferred embodiments of the invention support.

### DETAILED DESCRIPTION OF THE INVENTION

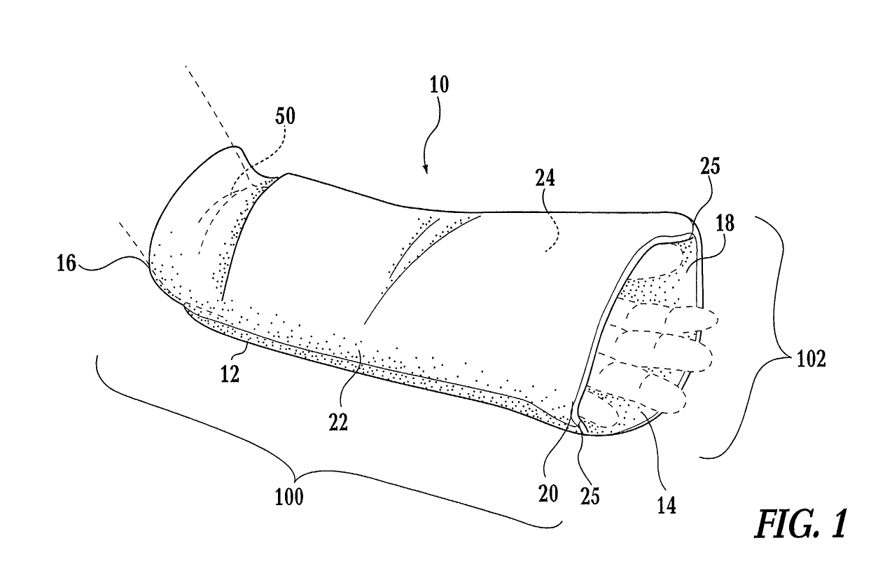

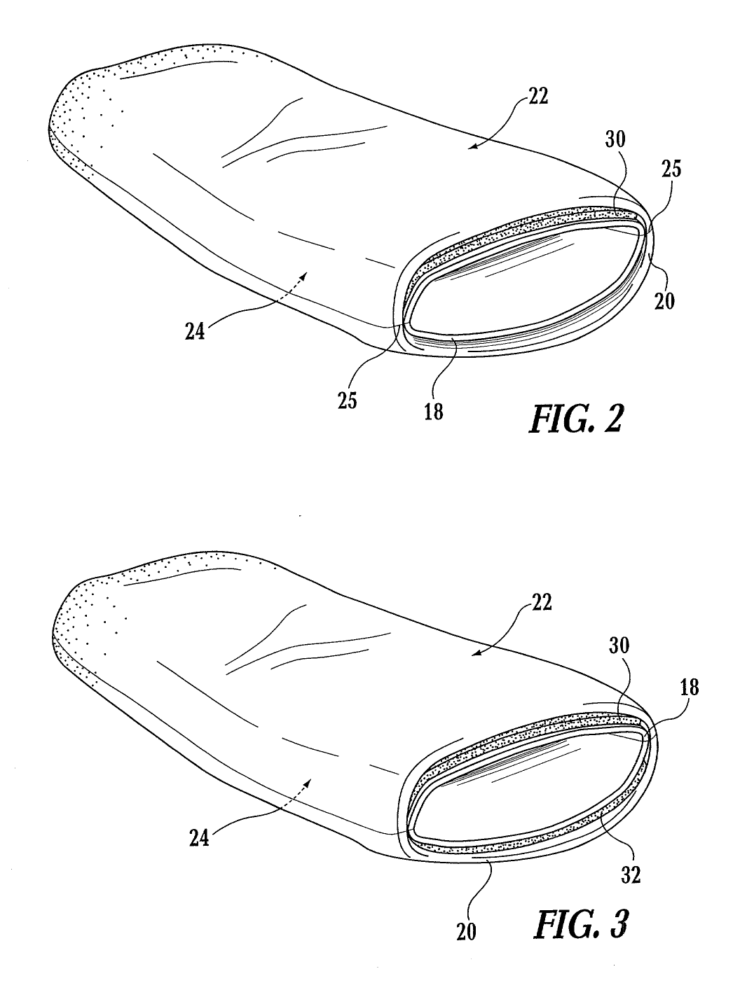

Referring to FIGS. 1 and 2, the breast and bottle feeding infant head support includes a slip-on sleeve 10 of a durable fabric material 12, preferably soft and absorbent, and/or washable, as a cotton fabric. The sleeve 10 is shown open at its opposite ends 14, 16 and includes inner and outer linings 18, 20 which form first and second opposing face surfaces 22, 24 of predetermined length 100 and width 102. The outer lining 20 may be sewn together along its length, and the inner lining 18 sewn to it as by tacking at 25. Between the linings 18, 20, a cushion insert 30 is included, of a resilient material such as a padded foam. As more clearly shown in FIG. 2, such insert is emplaced between the inner and outer linings 18, 20 beneath the face surface 22. Such insert 30 may be of a length substantially equal to the predetermined length 100 of the slip-on sleeve 10, and of a width substantially equal to that of the predetermined width 102. Alternatively, and where desired, the length of the cushion insert 30, on the other hand, may be selected somewhat less than the length 100 of the face surface 22. In either event, the slip-on sleeve 10, when worn on the arm (as shown) is of a length to extend past the crook of a wearer's elbow, shown at 50.

When worn in such manner, the infant's head can be supported along the forearm, or at the crook of the elbow at 50, resting on the surface 22 beneath which the cushion insert 30 sits. In such manner, a comfortable support for the head is provided, and the fabric of which the sleeve 10 is composed absorbs any perspiration that may be present. The resilient nature of the cushion cuddles the newborn and any tendency to flatten the head over time is reduced by virtue of the padded effect.

FIG. 3 illustrates the addition of a second cushion insert 32 between the inner and outer linings 18, 20 beneath the opposite face surface 24. Of a comparable length to the cushion insert 30, the second cushion insert 32 could likewise be of a length substantially equal to the length 100 of the face surface 24 in this respect, and of a width 102 substantially equal to the width of the face surface. If of a length somewhat less than the length 100, the second cushion insert 32 will fall short of reaching towards the palm of the wearer's hand, whose fingers could then encircle the fabric end of the sleeve 10. With such a construction, the slip-on sleeve 10 could be worn with the face surface 22 supporting the infant's head on one occasion, and then the face surface 24 provides the support, when the sleeve 10 is rotated in wearing.

With either configuration or arrangement, the overall result is to provide the comfort for the mother and child, cushioning the baby's head, absorbing any perspiration that may be present, and providing a sleeve which is washable and durable, and which is comfortable both for the infant and the mother too, in the breast and bottle feeding of her baby.

While there have been described what are considered to be preferred embodiments of the present invention, it will be readily appreciated by those skilled in the art that modifications can be made without departing from the scope of the teachings herein. For at least such reason, therefore, resort should be had to the claims appended hereto for a true understanding of the scope of the invention.

I claim:

1. A breast and bottle feeding infant head support comprising:
a slip-on sleeve open at opposite ends thereof and inclusive of inner and outer linings forming first and second opposing face surfaces of predetermined length and width;
and a cushion insert between said inner and outer linings beneath one of said opposing face surfaces; and
wherein said sleeve, when worn on the arm, is of a length to extend past the crook of a wearer's elbow.

2. The infant head support of claim 1 wherein said slip-on sleeve is of a durable fabric material.

3. The infant head support of claim 1 wherein said slip-on sleeve is of a soft absorbent fabric material.

4. The infant head support of claim 3 wherein said slip-on sleeve is of a washable material.

5. The infant head support of claim 4 wherein said slip-on sleeve is a cotton fabric.

6. The infant head support of claim 3 wherein said cushion

7. The infant head support of claim 3 wherein said cushion insert is of a foam padded material.

8. The infant head support of claim 6 wherein said cushion insert is of a length substantially equal to said predetermined length of said first and second opposing face surfaces.

9. The infant head support of claim 6 wherein said cushion insert is of a width substantially equal to said predetermined width of said first and second opposing face surfaces.

10. The infant head support of claim 6 wherein said cushion insert is of a length less than said predetermined length of said first and second opposing face surfaces.

11. The infant head support of claim 5, including a second cushion insert between said inner and outer linings beneath the other of said opposing face surfaces.

12. The infant head support of claim 11 wherein said second cushion insert is of a resilient material.

13. The infant head support of claim 11 wherein said second cushion insert is of a foam padded material.

14. The infant head support of claim 13 wherein said second cushion insert is of a length substantially equal to said predetermined lengths of said first and second opposing face surfaces.

15. The infant head support of claim 13 wherein said second cushion insert is of a width substantially equal to said predetermined width of said first and second opposing face surfaces.

16. The infant head support of claim 13 wherein said second cushion insert is of a length less than said predetermined lengths of said first and second opposing face surfaces.
